(19) United States
(12) Plant Patent Application Publication
(10) Pub. No.: US 2002/0152522 P1
(43) Pub. Date: Oct. 17, 2002

(54) PLUM TREE NAMED 'SUPLUMTWENTYTHREE'

(76) Inventors: Bruce D. Mowrey, La Selva Beach, CA (US); David W. Cain, Bakersfield, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/793,065

(22) Filed: Feb. 26, 2001

(51) Int. Cl. A01H 5/00

(52) U.S. Cl. PLT/184

(57) ABSTRACT

A new and distinct plum variety that possesses early ripening fruit which ripens evenly and has a mildly sweet taste is described. The fruit is not prone to fruit drop and has good keeping quality for the fresh market.

### BACKGROUND AND SUMMARY OF THE INVENTION

This invention relates to the discovery and asexual propagation of a new variety of plum, Prunus salicina cv. ‘Suplumtwentythree’. The new variety was first hybridized by Bruce D. Mowery. The new variety ‘Suplumtwentythree’ was selected and evaluated by David W. Cain near Wasco, Kern County, Calif. The variety was originated by open pollination of an unnamed, unpatented Sun World international breeding plum selection, designated as 91P-001. The new variety is characterized by its early ripening fruit, which ripens evenly and has a mildly sweet taste. The fruit is not prone to fruit drop and has good keeping quality for the fresh market.

‘Suplumtwentythree’ has as its seed parent an unnamed, unpatented plum variety designated as 91P-001. The new plum variety cv. ‘Suplumtwentythree’ arose from an open pollination of the seed parent and thus the pollen parent is an unknown plum variety. The parent varieties were first crossed in 1993, with the date of sowing of November 1993 and first flowering being in 1996. The new ‘Suplumtwentythree’ variety was first asexually propagated by David W. Cain in May 1998, near Wasco, Kern County, California, by budding.

‘Suplumtwentythree’ differs from its seed parent, 91P-001, by producing fruits that are slightly larger than its parent, averaging 6.2 cm diameter while 91P-001 averages 5.5 cm diameter. Fruits of the new variety cv. ‘Suplumtwentythree’ have red-colored flesh, while the flesh of 91P-001 is yellow. Moreover, ‘Suplumtwentythree’ ripens approximately four days after its seed parent 91P-001. The new variety ‘Suplumtwentythree’ closely resembles its sibling ‘Suplumtwentytwo,’ but ripens approximately fourteen days after ‘Suplumtwentytwo’.

‘Suplumtwentythree’ most nearly resembles the interspecific tree ‘Flavorosa’ variety (U.S. Plant Pat. No. 10,285). It is distinguished from the ‘Flavorosa’ variety by possessing more rounded elliptic leaves with less pronounced pointed lanceolate apices. The new variety is further differentiated from ‘Flavorosa’ by not being an interspecific hybrid with Prunus armeniaca parentage. ‘Suplumtwentythree’ fruits have flesh that tends to be lighter red near the pit cavity when commercially ripe as compared to the darker red coloration near the pit cavity in the ‘Flavorosa variety’. Additionally, the fruits of the new variety are able to hold more tenaciously to the tree, resulting in fruit that is less prone to fruit drop as the fruit approach maturity, in contrast to the ‘Flavorosa’ variety. Fruits of the new variety ripen approximately fourteen days after ‘Flavorosa’.

The flesh of ‘Suplumtwentythree’ fruit is red, in contrast to the yellow flesh of the ‘Black Beaut’ (U.S. Plant Pat. No. 3,617, expired) variety, and ripens approximately 5 days before ‘Black Beaut’. The new plum variety cv. ‘Suplumtwentythree’ ripens approximately 14 days after the ‘Red Beaut’ (U.S. Plant Pat. No. 2,539, expired) variety.

The new plum variety cv. ‘Suplumtwentythree’ has been shown to maintain its distinguishing characteristics through successive asexual propagations by, for example, budding.

### BRIEF DESCRIPTION OF THE FIGURE

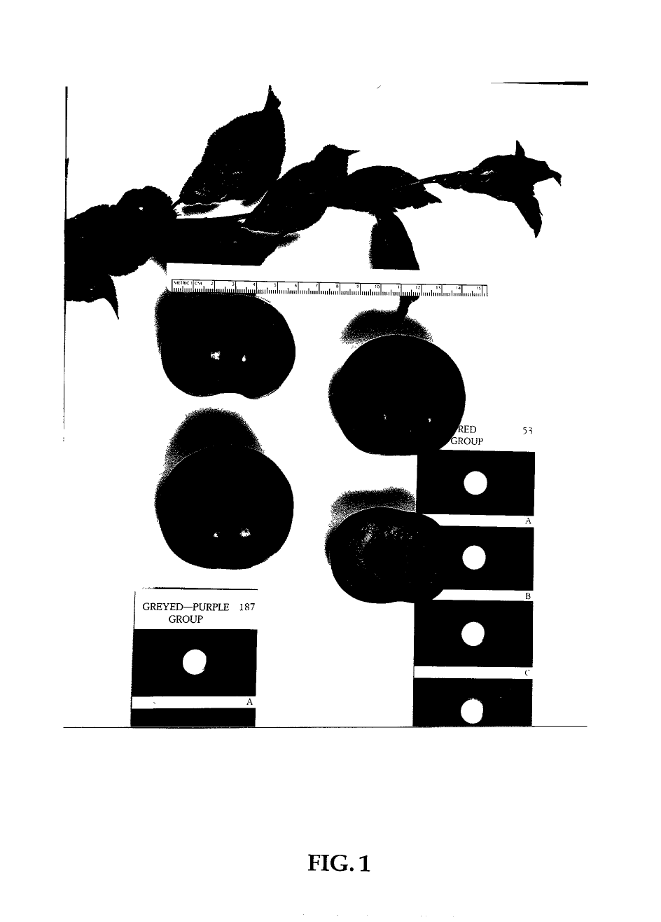

The accompanying drawing in FIG. 1 illustrates in full color.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon the R.H.S. Colour Chart, published by The Royal Horticultural Society, London, England.

The descriptive matter which follows pertains to ‘Suplumtwentythree’ plants grown in the vicinity near Wasco, Kern County, Calif., during 2000, and is believed to apply to plants of the same variety grown under similar conditions of soil and climate elsewhere:

### TREE

General:

Size.—medium.

Vigor.—medium.

Habit.—semi-upright.

Density of Foliage.—medium.

Shape.—vase formed.

Hardiness.—hardy in Kern County, California.

Productivity.—productive.

Fruit bearing.—regular bearer.

Root stock.—Flordaguard.

Trunk:

Shape.—medium.

Surface texture.—medium.

Branches:

Shape.—medium.

Surface texture.—medium.

Surface appearance.—semi-glossy.

Lenticels:

Number.—few.

Size.—medium.

### LEAVES

General:

Average length.—about 10.2 cm.

Average Width.—about 4.4 cm.

Size.—large—medium.

Outline.—broad obovate to elliptical.

Profile.—up folded.

Leaf blade tip.—curved downwardly.

Angle of tip.—acute to right angle.

Margin.—crenate.

Undulation of margin.—medium.

Apex.—cuspidate.

Base.—V-shaped.

Thickness.—medium.

Upper Surface:

Color.—about 147A.

Glossiness.—weak.

Pubescense.—absent.

Surface texture.—smooth.

Surface appearance.—dull.

Lower Surface:

Color.—about 147B.

Glossiness.—weak.

Pubescense.—absent.

Surface texture.—smooth.

Surface appearance.—dull.

Petiole:

Length.—medium, about 1 cm.

Thickness.—medium, about 2 mm.

Glands:

Average number.—3.

Positioning.—(a) on both leaf base and petiole; (b) alternate.

Size.—medium.

Shape.—Globose.

Color.—about 147A, becoming about Red-Purple 59A as it ages.

Stipules:

Persistence.—persistent.

Wood (Leaf) Buds:

Shape.—conical.

Size.—medium.

Position relative to shoot.—held out.

Support.—not decurrent.

Size.—medium.

Time of bud burst.—medium.

### FLOWERING SHOOTS:

Anthocyanin coloration.—present.

Intensity.—medium.

Thickness.—medium, about 0.47 cm.

Internode length.—medium, about 1.9 cm.

Density of buds.—medium.

Distribution.—isolated in groups of 2 or more on spurs and one year old shoots and older wood.

Ratio of wood (leaf) buds to flowering shoots.—1:4 (ranges from 1:4 to 1:6).

### FLOWERS

Flower-Buds:

Hardiness.—hardy.

Size.—medium.

Length.—medium.

Shape.—pointed—plump.

Positioning.—free.

Pubescence.—absent.

Color.—about 177A.

General:

Date of first bloom.—Mar. 3, 2000.

Date of full bloom.—Mar. 8, 2000.

Time of bloom.—medium, as compared with similar varieties in the growing area of Wasco, Kern County, Calif.

Duration of bloom.—medium, about 10 days.

Size (diameter of the fully open flower).—medium, about 2.1 cm.

Shape.—rosaceous.

Petals.—free.

Color.—(fully opened flower): White.

Peduncle:

Length.—medium, about 0.7 cm.

Thickness.—medium.

Pubescence.—absent.

Receptacle:

Depth.—medium.

Pubescence of inner surface (at white bud stage).—absent.

Pubescence of outer surface.—absent.

Sepals:

Positioning.—adpressed to petals.

Shape.—ovate.

Pubescence of inner surface.—absent.

Pubescence of outer surface.—absent.

Frequency of flowers with double sepals.—none.

Petals:

Frequency of flowers with double petals.—none.

Size.—medium, about 0.8 cm. width.

Shape.—transverse broad elliptic.

Claw length.—short.

Margin waviness.—medium.

Base angle.—medium.

Division of upper margin.—entire.

Pubescence of inner surface.—absent.

Pubescence of outer surface.—absent.

Color (inner surface).—White.

Color (outer surface).—White.

Stigma:

Position (as compared with anthers).—level.

Anthers:

Color (just before dehiscence).—about Yellow 23B with slight shadings of about Red 45D.

Pollen:

Color.—about 13A.

Stamens:

Position.—perigynous.

Pistil:

Frequency of supplementary pistils.—absent.

Number.—always one.

Ovary:

Pubescence.—absent.

Style:

Pubescence (of base).—absent.

### FRUIT

General:

Maturity when described.—firm—ripe.

Date.—Jun. 3, 2000.

Size.—uniform; medium-large; about 6.2 cm.

Axial Diameter.—about 4.7 cm.

Transverse in suture plane.—about 5.9 cm.

At right angles to suture plane.—about 6.2 cm.

Form.—oblate—rounded.

Position of maximum diameter.—towards middle.

Symmetry about the suture.—nearly symmetric.

Season ripening.—early.

Use.—fresh market.

Keeping quality.—good.

Resistance to.—Insects: medium; typical of Japanese type plums. Diseases: medium; typical of Japanese type plums. Shipping quality: medium.

Suture: an inconspicuous line, the suture is shallow and extends from base to apex.

Ventral Surface:

Shape.—rounded slightly.

Lips.—equal.

Depression of apex.—slightly—depressed.

Pistil base.—not persisting.

Pubescence at apex.—absent.

Stem Cavity:

Shape.—rounded and slightly elongated in suture plane sides.

Depth.—about 0.8 cm.

Breadth.—about 1.1 cm.

Markings.—none.

Base:

Shape.—rounded—truncate.

Apex:

Shape.—truncate and slightly depressed.

Pistil Point:

Shape.—slightly depressed.

Stem:

Length.—medium, about 1.5 cm.

Adherence to stone.—strong—medium

Skin:

Thickness.—medium.

Texture.—medium.

Reticulation.—absent.

Roughness.—absent.

Taste.—slightly tart.

Tenacity.—tenacious to flesh.

Tendency to crack.—none in wet—dry season.

Color.—about 187A, becoming black as it ripens.

Down (pubescence).—none.

Bloom.—present.

Flesh:

Color.—about Red 53A; lighter colored near the stone (about 48C) if not fully mature.

Surface of the pit cavity.—Slightly rough.

Amygdalin.—wanting.

Juice.—abundant to moderate.

Sugar content.—medium (about 15% SS).

Texture.—medium, fine, melting.

Fibres.—medium, fine, tender.

Ripens.—even.

Flavor.—mildly sweet.

Aroma.—distinct.

Eating Quality.—good.

Stone/flesh ratio.—1.7 g/115 g.

### STONE

General:

Adherence to flesh.—cling.

Fibres.—retains short fibre.

Size.—medium.

Length.—about 2 cm.

Breadth.—about 1.9 cm.

Width of stalk end.—about 0.2 cm.

Angle of stalk end.—right angle to obtuse.

Thickness.—about 1.2 cm.

Form (profile).—globose-elliptical.

Form (ventral view).—sub-globular.

Base.—nearly straight.

Hilum.—narrow.

Apex.—obtuse with a small sharp point.

Position of maximum breadth.—middle, but varies with the growth of the keel.

Sides.—nearly equal.

Surface.—irregularly furrowed near base, rough but not heavily pitted.

Outgrowing keel.—well developed.

Ridges.—jagged.

Pits.—angular.

Ventrical edge.—thin with wing throughout.

Dorsal edge.—medium width with distinct shallow groove throughout.

Color of stone.—about 164B.

Tendency to split.—slight in wet/dry season.

Suplumtwentythree: Table of R.H.S. Color Codes:

| | | | R.H.S. Color Code |
|---|---|---|---|
| LEAVES: | Upper Surface: | | 147A |
| | Lower Surface: | | 147B |
| | Glands: | Early: | 147A |
| | | Upon aging: | 59A |
| FLOWERS: | Flower Buds: | | 177A |
| | Anthers: | Just before | 23B with slight |
| | | dehiscence: | shadings of 45D |
| | Pollen: | | 13A |
| FRUIT: | Skin: | | 187A (becoming |
| | | | black as it ripens) |
| | Flesh: | General color: | 53A |
| | | Color near stone | 48C |
| | | if not fully mature: | |
| STONE: | | Stone color: | 164B |

What is claimed is:

1. A new and distinct variety of plum cv. ‘Suplumtwentythree’ as herein illustrated and described.
